(19) United States
(12) Patent Application Publication
(10) Pub. No.: US 2012/0052054 A1
(43) Pub. Date: Mar. 1, 2012

(54) METHOD FOR PREVENTING SKIN ELASTICITY LOSS BY SUPPRESSING INCREASE OF SUBCUTANEOUS FAT

(75) Inventor: Tomonobu Ezure, Kanagawa (JP)

(73) Assignee: SHISEIDO COMPANY, LTD., Tokyo (JP)

(21) Appl. No.: 13/264,199

(22) PCT Filed: Apr. 13, 2010
(86) PCT No.: PCT/JP2010/056617

§ 371 Date: Oct. 13, 2011

Related U.S. Application Data
(60) Provisional application No. 61/168,704, filed on Apr. 13, 2009.

(51) Int. Cl. A61K 8/66; A61F 7/00; A61Q 19/00

(52) U.S. Cl. 424/94.64; 607/96

(57) ABSTRACT

A cosmetic method for preventing an aggravation of skin condition accompanied with elasticity loss such as sag and a wrinkle is developed. The invention provides the method for preventing an elastic property loss specifically including the step of suppressing the increase in subcutaneous fat. In the step of the present invention for preventing the elastic property loss, the step of suppressing the increase in the subcutaneous fat may include a step of a thermal stimulation. In the step of the present invention for preventing the elastic property loss, the step may include a step of administering to a subject a composition which suppresses an increase in subcutaneous fat. The present invention provides a cosmetic method for preventing an aggravation of skin condition accompanied with elasticity loss, specifically including a step of applying to a skin a method for preventing the elastic property loss of skin.

### FIELD OF THE INVENTION

The present invention relates to a cosmetic method, more particularly to a method for preventing a skin elastic property loss by suppressing an increase of a subcutaneous fat.

### BACKGROUND ART

A skin is constituted from an epidermis, a dermis, a subcutaneous tissue and the like. The skin also serves as a supportive tissue which supports the inside of a body, and its physical properties are important for defending against an external physical stimulation, keeping an internal tissue in place and the like. A deterioration in such a physical property of the skin, especially skin viscoelasticity, due to a certain cause is known to lead to an aggravation of sagging of the skin (Non-Patent Document 1). While the effects of ultraviolet rays and aging have been studied conventionally with regard to a skin viscoelasticity loss, an effect by a subcutaneous fat has not been known. For example, Non-Patent Document 2 presents a hypothesis that “Based on the results indicating that a thicker subcutaneous fat of a face tends to give a lower level of sagging, a subcutaneous fat in a face part gives a plump appearance and a tension to the skin whereby allowing a strained shape, which serves to suppress sagging. However, a subcutaneous fat in a trunk part, amount of which is greater by 10 times than that in a face part, can no longer be supported by the skin and sags down due to the gravity, thus forming a sagging.”

### PRIOR ART DOCUMENTS

### Non-Patent Documents

Non-Patent document 1: Ahn, S. et al., Skin Research and Technology, 13:280-284.
Non-Patent document 2: Murakami, M. et al., “KOSHOKAISHI” (Journal of Japanese Cosmetic Science Society), 21:190-196.

### DISCLOSURE OF THE INVENTION

### Problem to be Solved by the Invention

While an aggravation of the skin condition accompanied with a skin viscoelasticity loss such as a sag and a wrinkle is of a great cosmetic concern, there was almost no effective method for preventing it so far. An object of the present invention is to develop a cosmetic method for preventing an aggravation of the skin condition accompanied with a skin elasticity loss such as a sag and a wrinkle based on an interaction between a subcutaneous fat and a dermis.

### Means for Solving the Problem

We found that the skin elastic property is reduced as the subcutaneous fat is increased. We also found that, in a dermis layer whose subcutaneous fat is increased a matrix metalloproteinase (MMP) is increased, whereby reducing the number of fibroblasts. We further found that a hypertrophic fat cell suppresses not only the increase in the fibroblasts but also the production of an extracellular matrix component by the fibroblasts. Such findings indicate that an increase in the subcutaneous fat leads to a reduction in the extracellular matrix components such as collagen, elastin, hyaluronic acid which constitute a dermis layer, especially an extracellular matrix of the dermis layer, resulting in a skin viscoelasticity loss. Accordingly, we achieved the present invention which relates to a method for preventing a skin elastic property loss by suppressing an increase of a subcutaneous fat, and a cosmetic method for preventing an aggravation of the skin condition accompanied with a skin viscoelasticity loss by applying the said method to a skin.

The present invention provides a method of preventing a skin elastic property loss comprising a step of inhibiting an increase in a subcutaneous fat.

In the method for preventing a skin elastic property loss according to the present invention, the step of inhibiting an increase in the subcutaneous fat may comprise a step of applying a hyperthermic stimulation.

In the method for preventing a skin elastic property loss according to the invention, the step of inhibiting an increase in the subcutaneous fat may comprise a step of administering to a subject a composition which inhibits an increase in a subcutaneous fat.

The present invention provides a cosmetic method for preventing an aggravation of a skin condition accompanied with a skin elastic property loss by applying to a skin the method for preventing a skin elasticity loss according to the present invention.

The present invention provides a method for preventing a reduction in an extracellular matrix component in a dermis layer comprising a step of inhibiting an increase in a subcutaneous fat.

In the method for preventing a reduction in an extracellular matrix component in a dermis layer according to the invention, the step of inhibiting an increase in a subcutaneous fat may comprise a step of applying a hyperthermic stimulation.

In the method for preventing a reduction in an extracellular matrix component in a dermis layer according to the present invention, the step of inhibiting an increase in the subcutaneous at may comprise a step of administering to a subject a composition which inhibits an increase in a subcutaneous fat.

The invention provides a cosmetic method for preventing an aggravation of the skin condition accompanied with a skin elasticity loss by applying to a skin the method for preventing a reduction in an extracellular matrix component in a dermis layer according to the present invention.

In the method for preventing a reduction in an extracellular matrix component in a dermis layer according to the invention, the extracellular matrix component may be at least one of collagen, elastin and hyaluronic acid.

The invention provides a cosmetic method for preventing a wrinkle and a sag, comprising applying to a skin the method for preventing a reduction in an extracellular matrix component in a dermis layer according to the invention.

The invention provides a composition for preventing an aggravation of the skin condition accompanied with a skin elasticity loss, comprising a composition which suppresses an increase in the subcutaneous fat.

As used herein, a “wrinkle” refers to a kind of skin trouble, and is a state in which patterns formed from linear recesses on the skin surface are concentrated on a particular region where they exist with an irregularity in size and alignment.

As used herein, a “sag” refers to a kind of skin trouble, and is a state in which the skin tension is lost and a swollen skin is observed on the entire face including a periocular region, a perioral region, and lower cheeks, jaw, neck and the like.

As used herein, a “matrix metalloproteinase (MMP)” refers to an enzyme belonging to a MMP family which binds to a metal, especially to zinc, and has an activity for cleaving most of the extracellular matrix constituents. Twenty five or more of members of the MMP family have been identified.

The amino acid and polynucleotide sequences of an MMP family member enzyme can be searched in the United State NCBI website (http://www.ncbi.nlm.nih.gov/sites/gquery) which contains OMIM (trademark, Online Mendelian Inheritance in Man) database. Among the MMP family members, MMP2 and MMP9 are enzymes which mainly break down type IV collagen. MMP3 is an enzyme which breaks down not only type IV collagen but also proteoglycan, fibronectin and laminin. MMP12 is an enzyme which breaks down an insoluble elastin. MMP13 is an enzyme which breaks down type II collagen contained in a cartilage. MMP14 is an enzyme which cleaves a precursor of MMP 2. MMP2, MMP3, MMP9 and MMP14 are expressed in a white fat cell, and it is reported that, among these, an MMP14 gene knockout mouse exhibits a white fat cell differentiation disorder (Chun, T-H. et al., Cell, 125:577-591).

A step of suppressing an increase in the subcutaneous fat according so the present invention can be accomplished by various means including, but not limited to, dietary restriction, exercise, hyperthermic stimulation, administration of a composition suppressing an increase in the subcutaneous fat to a subject.

The hyperthermic stimulation according to the present invention refers to a hyperthermic stimulation under any condition which suppresses an increase in the subcutaneous fat. It is preferable to allow a subcutaneous fat to be subjected to a hyperthermic stimulation at 41 to 43° C. for 30 to 90 minutes, and it is more preferable to allow a subcutaneous fat to be subjected to a hyperthermic stimulation at 41.5 to 43° C. for 60 minutes. The hyperthermic stimulation is fully described in the specification of U.S. patent application Ser. No. 12/253,758 filed by us to the Patent and Trademark Office on Oct. 17, 2008, which is hereby incorporated by reference in its entirety.

A composition which suppresses an increase in the subcutaneous fat according to the present invention includes, but is not limited to, a adiposity inhibitor, a lipid synthesis inhibitor, an anorectic agent, a fat cell differentiation inhibitor, a fat cell proliferation inhibitor, a fat metabolism modifier and the like. The adiposity-inhibitor includes, but is not limited to, a lipase inhibitor produced in a pancreas (Japanese Unexamined Patent Application Publication No. 2001-226274), an elastase and the like which promote degradation and excretion of hepatic and blood triglycerides. The lipid synthesis inhibitor includes, but is not limited to, an HMG-CoA reductase inhibitor such as pravastatin sodium, and a fibrate-based agent which acts on an intranuclear receptor PPAR-α to control the synthesis of a protein involved in a lipid synthesis. The anorectic agent includes, but is not limited to, mazindol, leptin and the like. The fat cell differentiation inhibitor includes, but is not limited to, the extracts of madder plant, sweet hydrangea leaf and the like (Japanese Unexamined Patent Application Publication No. 2002-138044). The at cell proliferation inhibitor includes, but is not limited to, dihomo-γ-linolenic acid (Japanese Unexamined Patent Application Publication No. 2006-306813). The fan metabolism modifier includes, but is not limited to, a thiazolidine-based insulin sensitizer or the like such as pioglitazone.

In the present invention, measurements of the thicknesses of the dermis layer, the subcutaneous fat and the cutaneous muscle, the elastic properties of the skin, the quantities of the dermis extracellular matrix components, the matrix metalloproteinase level and the number of fibroblasts may be carried out using any measuring methods known to those skilled in the cosmetic art.

All references cited herein are hereby incorporated by reference in their entirety.

### BRIEF DESCRIPTION OF THE DRAWINGS

FIG. 1 is a graph showing the thickness of the dermis layer in the tissue specimens of dorsal skins.

FIG. 2 is a graph showing the thickness of the cutaneous muscle part in the tissue specimens of dorsal skins.

FIG. 3 is a graph showing the thickness of the dermis layer in the auricular tip skin region after the end of the feeding period.

FIG. 4 is a graph showing the numbers of dermis cells per unit area of a dorsal skin.

FIG. 5 is a graph showing the expression levels of various MMP family genes in skin tissues in the treatment and the control groups.

FIG. 6A is a general pattern of cutometry deformation curve.

FIG. 6B is a graph of Uf and Ua when compared between the high fat diet treatment group hairless mice (HFD) and the control group hairless mice (Control).

FIG. 6C is a graph of Ua/Uf, Ur/Uf and Ur/Ue when compared between the high fat diet treatment group hairless mice (HFD) and the control group hairless mice (Control).

FIG. 7A is a photograph showing a lower cheek skin of a human in a sitting-up position.

FIG. 7B is a photograph showing a lower cheek skin of a human in a supine position.

FIG. 7C is an ultrasonic tomogram of the skin of a lower cheek part.

FIG. 8A is a graph showing the correlation between a skin viscoelastic property (Ue) and the subcutaneous fat layer thickness.

FIG. 8B is a graph showing the correlation between a skin viscoelastic property (Ur) and the subcutaneous fat layer thickness.

FIG. 8C is a graph showing the correlation between a skin viscoelastic property (Uf) and the subcutaneous fat layer thickness.

FIG. 8D is a graph showing the correlation between a skin viscoelastic property (Ua) and the subcutaneous fat layer thickness.

FIG. 8E is a graph showing the correlation between a skin viscoelastic property (Ua/Uf) and the subcutaneous fat layer thickness.

FIG. 8F is a graph showing the correlation between a skin viscoelastic property (−Uv/Ur) and the subcutaneous fat layer thickness.

FIG. 9A is a graph showing the correlation between a skin viscoelastic property (Ur) and the age.

FIG. 9B is a graph showing the correlation between a skin viscoelastic property (−Uv) and the age.

FIG. 9C is a graph showing the correlation between a skin viscoelastic property (−(Uf−Ua)) and the age.

FIG. 9D is a graph showing the correlation between a skin viscoelastic property (Ua/Uf) and the age.

FIG. 9E is a graph showing the correlation between a skin viscoelastic property (Ur/Ue) and the age.

FIG. 9F is a graph showing the correlation between a skin viscoelastic property (Ur/Uf) and the age.

FIG. 10A is microscopic photographs of 3T3-L1 cells on the 10th day (left) and the 25th day (right) after differentiation induction as being stained with Oil Red O.

FIG. 10B is a graph showing the change in the lipid-accumulation after starting the differentiation induction in the 3T3-L1 cells.

FIG. 10C is a schematic view of the experiment of culturing together.

FIG. 11 is a graph showing the effect of the 3T3-L1 cell induced differentiation into fat cells on the proliferation of the fibroblasts during culturing together.

FIG. 12 is a graph showing the effect of the 3T3-L1 cells induced differentiation into fat cells on the collagen production by the fibroblast during culturing together.

FIG. 13 is a graph showing the effect of the 3T3-L1 cell induced differentiation into fat cells on the elastin production by the fibroblasts during culturing together.

FIG. 14 is a graph showing the effect of the 3T3-L1 cell induced induction into fat cells on the hyaluronic acid production by the fibroblasts during culturing together.

### DESCRIPTION OF EMBODIMENTS

Examples of the present invention described below are intended only to exemplify the invention rather than to limit the technical scope thereof. The technical scope of the present invention is limited only by the description in claims.

The researches described in the following examples were carried out after approval by the Ethics Committee of the Shiseido Research Center in accordance with National Institutes of Health (NIH) of the United States.

### Example 1

### Measurements of Numbers of Cells at the Dermis Layer and Subcutaneous Fat in Dorsal Skin

Materials and Methods

Hairless mice (HR-1, Males, six-week old, Hoshino Laboratory Animals, Inc.) were employed. In a treatment group, six hairless mice were fed with a high fat diet (containing 30% lipid, Oriental Yeast Co., Ltd.) for 12 weeks to investigate the effect of the high fat diet on the thickness of the dermis layer. In a control group, six hairless mice were fed with an ordinary feed for 12 weeks. After the end of the feeding period, the dorsal and auricular tip skins were obtained from the hairless mice in the treatment and control groups. The skins were fixed in 10% formalin, embedded in paraffin, sliced into sections, which were then stained with hematoxylin-eosin (HE). The production and the HE stainings of the sections were carried out according to a conventional method known to those skilled in the art.

Results

Histological Findings of Dorsal Skin

From the dorsal skin obtained after the end of the feeding period described above, sliced sections were prepared and HE-stained to obtain tissue specimens, which were observed by an optical microscope. When compared with the control group, she treatment group exhibited a dorsal skin tissue specimen in which a subcutaneous fat was increased and the dermis layer was markedly reduced. Based on these results, it was revealed that the dermis layer tended to be reduced as the subcutaneous fat was increased.

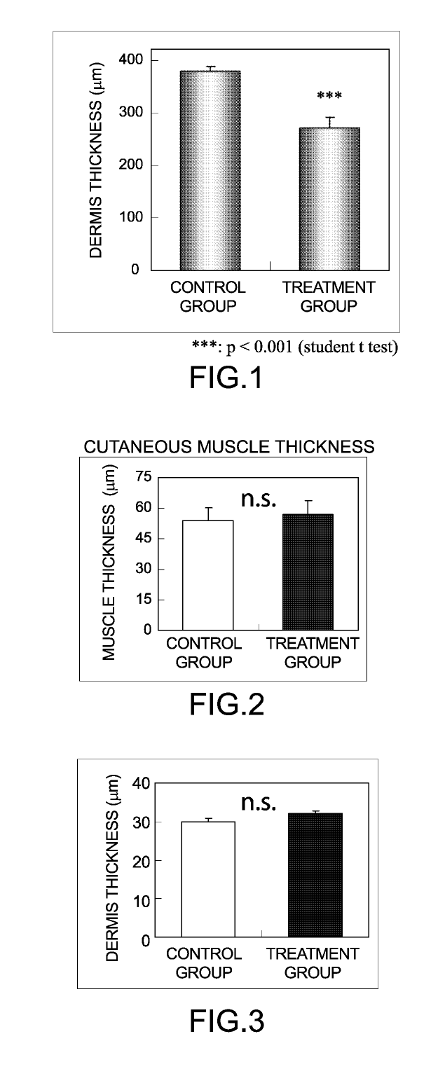

FIG. 1 is a graph showing the thickness of the dermis layer in the dorsal skin tissue specimen. The mean values of the thickness of the dorsal skin dermis layers of six individual animals in the treatment and control groups were 280 μm (micrometer) in the treatment group and 380 μm (micrometer) in the control group. The error bars in the graphs indicate the standard deviations of the measured thicknesses of the dermis layers of each 6 individual animals in the treatment and control groups. When conducting Student's t-test for the significant difference in the mean value between the treatment group and the control group indicated with asterisks (***) in the graph, the p value was revealed to be less than 0.1%. Accordingly, the reduction in the dermis layer in the treatment group when compared with the control group is statistically significant. Based on these results, it was revealed that in the dorsal skin the dermis layer was reduced markedly as the subcutaneous fat was increased.

FIG. 2 is a graph showing the thickness of the cutaneous muscle part in the dorsal skin tissue specimen. The mean values of the thickness of the dorsal skin cutaneous muscle parts of 6 individual animals in the treatment and control groups were 57 μm (micrometer) in the treatment group and 52 μm (micrometer) in the control group. The error bars in the graphs indicate the standard deviations of the measured thicknesses of the cutaneous muscle parts of each 6 individual animals in the treatment and control groups. There was no statistically significant difference (n.s.) in the mean value between the treatment group and the control group, and it was revealed that there was no difference in the thickness of the cutaneous muscle part between the treatment group and the control group. Similarly, there was no difference in the thickness of the epidermis layer between the treatment group and the control group (data not shown). Based on these results, the change in the thickness niche dorsal skin tissue is considered to be specific only to the dermis layer. Accordingly, the reduction in the dermis layer is considered not due to a physical extension resulting from the growth and the obesity of the individual mice fed with the high fat diet.

Histological, Findings of Auricular Tip Skin

In order to investigate the relationship between the marked reduction in the dorsal skin dermis layer and the increase in the subcutaneous fat, the mouse auricular tip tissue having no subcutaneous fat was studied. FIG. 3 is a graph showing the thickness of the dermis layer in the auricular tip skin of the end of the feeding period. The mean values of the thickness of the auricular tip dermis layers of 6 individual animals in the treatment and control groups were 32 μm (micrometer) in the treatment group and 30 μm (micrometer) in the control group. The error bars in the graphs indicate the standard deviations of the measured thicknesses of the dermis layers of each 6 individual animals in the treatment and control groups. There was no statistically significant difference (n.s.) in the mean value between the treatment group and the control group, and it was revealed that there was no difference in the thickness of the dermis layer of the auricular tip part having no subcutaneous fat between the treatment group and the control group.

Based on these histological findings, the possibility that the cause of the reduction in the dermis layer was the increase in the subcutaneous fat was suggested. Accordingly, the mechanism of the reduction in dermis layer was further studied.

### Example 2

### Measurements of Number of Cells in Dermis Layer

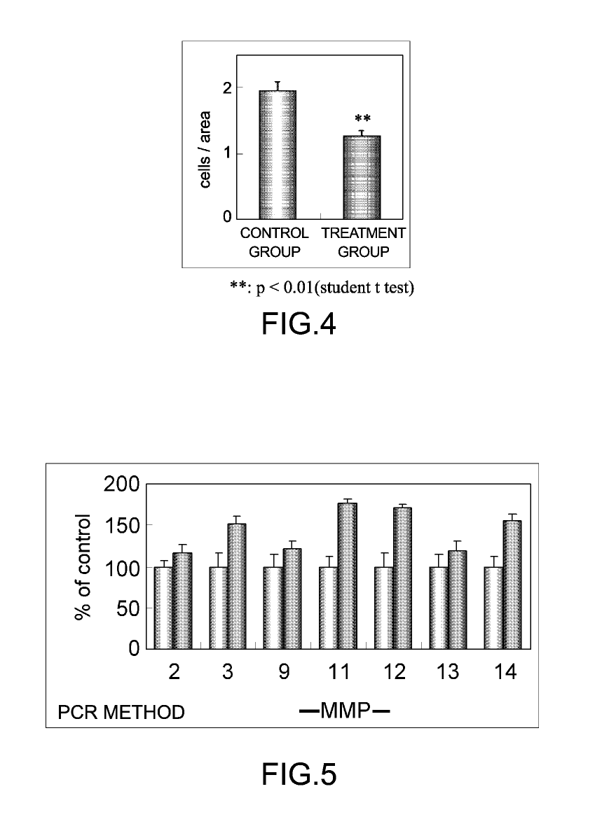

FIG. 4 is a graph showing the numbers of dermis cell per unit area of the dorsal skin described above. The mean dermis cell per unit area of the dorsal skin of 6 individual animals in the treatment and control groups were 1.3 and 2 cells, respectively. The error bars for relevant experimental conditions are the standard deviations of the numbers of dermis cell per unit area. When conducting Student's t-test for the significant difference in the mean value of the numbers of dermis cell per unit area between the treatment group and the control group indicated with asterisks (**) in the graph, the p value was revealed to be less than 1%. Accordingly, the reduction in the fibroblasts in the treatment group when compared with the control group is statistically significant. Based on these results, it was revealed that the number of fibroblast was reduced markedly as the subcutaneous fat was increased.

### Example 3

### Quantitative Analysis of Expression Level of Matrix Metalloproteinase Genes

Materials and Methods

After the end of the feeding period described above, the dorsal skin tissues were obtained from the hairless mice in the treatment and control groups, and mRNAs were extracted from these tissues according to a conventional method and cDNAs were synthesized. By a real-time PCR method using these cDNAs as templates, the expression levels of various MMP genes were determined. The expression levels of the MMP genes were normalized on the basis of the expression level of glyceraldehyde 3-phosphate dehydrogenase (GAPDH) gene with representing the expression level in the control group as 100%.

Results

FIG. 5 is a graph showing the expression levels of various MMP family genes in skin tissues in the treatment and control groups. In this example, the expression levels of MMP2, MMP3, MMP9, MMP11, MMP12, MMP13 and MMP14 genes were determined. As a result, when compared with the expression level in the control group, the relative expression level of MMP2 was 120%, the relative expression level of MMP3 was 150%, the relative expression level of MMP9 was 130%, the relative expression level of MMP11 was 180%, the relative expression level of MMP12 was 170%, the relative expression level of MMP13 was 120%, and the relative expression level of MMP14 was 150%. The error bars in the graphs indite the standard deviations of the measured expression levels of each six individual animals in the treatment and control groups. From the graph in FIG. 5, it was revealed that the expression level of every MMP gene was higher in the hairless mice fed with the high fat diet than in the hairless mice fed with the conventional feed. Based on these results, it was revealed that the dermis layer was reduced as the expression level of the MMP gene was increased.

When observing the dorsal tissue specimens using an optical microscope at a high magnification, it was recognized that the boundary between the dermis layer and the subcutaneous fat was relatively flat in the control group, while an image of the subcutaneous fat intruding into the dermis layer was observed in the treatment group (data not shown). Accordingly, it is suggested that the extracellular matrix of the dermis layer formed mainly from a collagen is broken down by an MMP whereby causing an irregularity in the laminar structure of the dermis which allows an infiltration of the subcutaneous fat cells.

### Example 4

### Measurements of Mouse Skin Viscoelastic Properties

A Cutometer MPA580 (trademark, Koln in Germany, Courage and Khazaka) which was a non-invasive viable skin viscoelasticity measuring device utilizing a negative pressure suction was employed. A mouse anesthetized with an intraperitoneal injection of pentobarbital was subjected to a dorsal skin suction for two seconds with a negative pressure of 50 mbar using a probe of two mm in diameter, followed by recovery to an atmospheric pressure for a 2-second relaxation period, and the skin recovery was recorded as a curve pattern. The skin viscoelasticity parameters were selected based on Deleixhe-Mauhin, F. et al., (Clin. Exp. Dermatol. 19: 130-133 (1994)).

Results

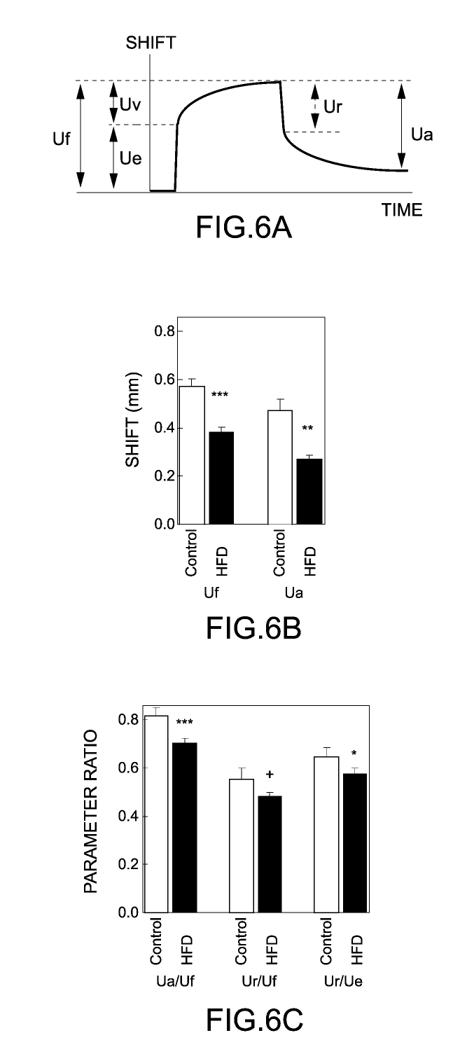

FIG. 6A is a general pattern of deformation curve of a cutometery measurement. The ordinate represents the relative value of the skin shift, while the abscissa represents the elapsing time. The curve protruding upward represents the deformation condition during the suction under a negative pressure, while the curve protruding downward represents the state of recovery of the skin once released from the negative pressure. Uf represents a maximum suction value (final distention), Ue represents a elastic deformation component (immediate distention), Uv represents a viscoelastic component (viscoelastic creep occurring after the elastic deformation), Ur represents a elasticity recovery component (immediate retraction), and Ua represents a value of complete recovery from deformation (final retraction).

FIG. 6B is a graph which compares Uf (maximum suction value) and Ua (value of complete recovery from deformation) between the hairless mice fed with the high fat diet for 12 weeks (HFD) and the hairless mice fed with the conventional feed for 1.2 weeks (Control) as described in example 1. FIG. 6C is a graph which compares Ua/Uf (total skin elasticity degree including viscoelastic deformation), Ur/Uf (biological elasticity degree) and Ur/Ue (total elasticity degree) between the treatment group hairless, mice fed with the high fat diet for 12 weeks (HFD) and the control group hairless mice fed with the conventional feed. For 12 weeks (Control). Based on FIGS. 6B and C, the skin elasticity parameters in the treatment group were significantly reduced than those in the control group. The error bars in the graphs indicate the standard deviations of the skin elasticity parameters of each six individual animals in the treatment and control groups. When conducting Student's t-test for the significant difference in the mean value between the treatment group and the control group indicated with symbols (+, *, ** and ***) in the graph, the p values were revealed to be less than 10%, less than 5%, less than 1% and less than 0.1%, respectively. Accordingly, the reduction in the parameters of the skin elasticity degree in the treatment group when compared with the control group is statistically significant.

### Example 5

### Measurements of Viscoelastic Properties of Human Facial Skin

Subjects

Healthy female volunteer subjects consisting of 17 in their thirties, 36 in their forties, and 17 in their fifties were recruited. These female subjects had BMI values of 17.1 to 36.2 kg/m² (kg/m .sup. 2), received no medication, had no history of surgical operations, were not cigarette smokers, and had no history of diabetes mellitus.

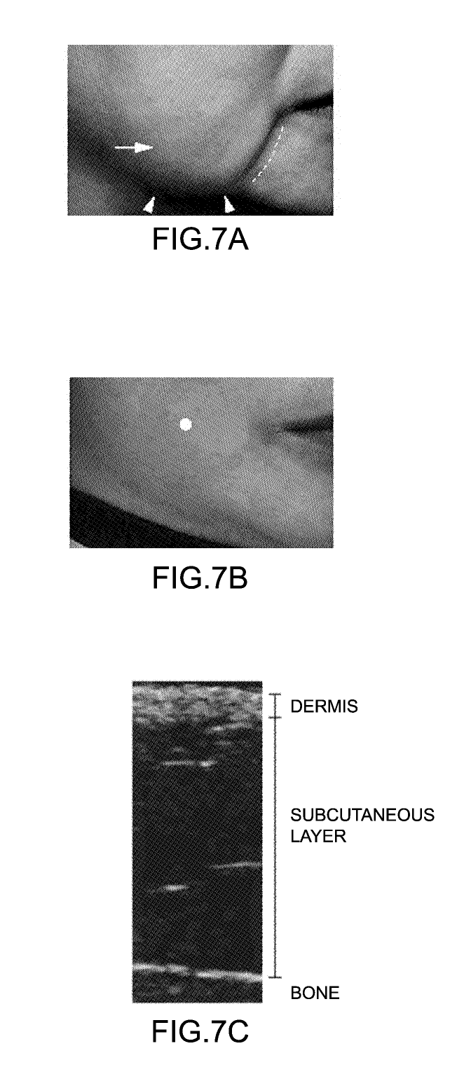

FIG. 7A is a photograph showing a lower cheek skin of a subject in a sitting position. The wavy line indicates a marionette line (a wrinkle resulting from a sag surrendering to gravity), the arrow indicates a protruding region of the cheek, and the arrowhead indicates the profile of the lower chin. FIG. 7B is a photograph showing a lower cheek skin of the same subject in a supine position with her neck tilted by 45 degrees to make the cheek horizontal. The white dot is the center of the cheek at a distance of 3 cm from the angle of the mouth. A sag was observed in the lower cheek skin sagging down due to the gravity to show a sag when the subject was in the sitting position (FIG. 7A), while no sag was observed when the same subject was in a supine position with her neck tilted by 45 degrees to make the cheek horizontal. Accordingly, the center of the cheek (white dot in FIG. 7B) was subjected to the skin viscoelastic property measurement by the Cutometer and to the subcutaneous fat layer thickness measurement by an ultrasonic tomography (echography) while the subject was in a supine position with her neck tilted by 45 degrees to make the cheek horizontal.

Skin Viscoelastic Property Measurements

The skin viscoelastic properties of the face was measured using the Cutometer employed in example 4. The procedures of the measurement were the same as those in example 4 except that the negative pressure applied was 400 mbar and the skin at the center of the cheek at a distance of three cm from the angle of the mouth was measured while the subject was in a supine position with her neck tilted by 45 degrees to make the cheek horizontal.

Face Subcutaneous Fat Thickness Measurement

While the subject was in a supine position with her neck tilted by 45 degrees to make the cheek horizontal, an echography gel was applied as a thin film onto the skin at the center of the cheek at a distance of three cm from the angle of the mouth, on which a 13 MHz probe of an ultrasonic tomography imaging device (Prosound alpha 5 (trademark), Aloka) was pressed vertically to the skin, whereby imaging the subcutaneous tissue in a B-mode. The subcutaneous fat layer thickness is defined as a distance from the bottom of a dermis to the top of the oral mucosa including a thin layer of the facial expression muscles.

Results

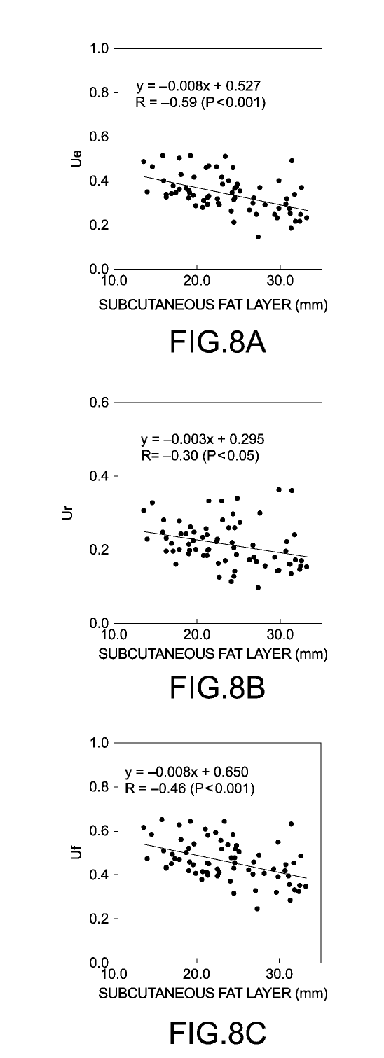

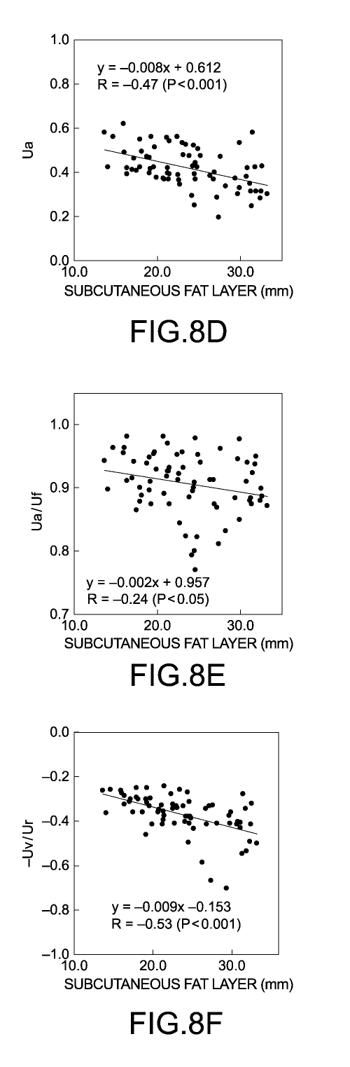

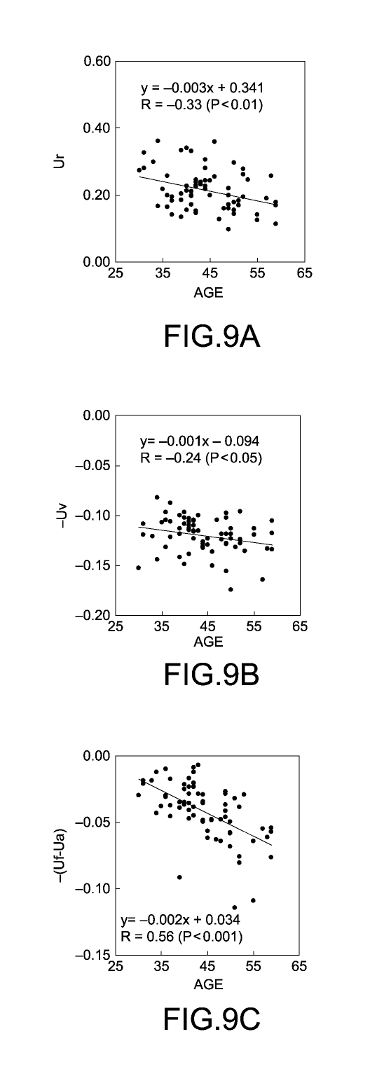

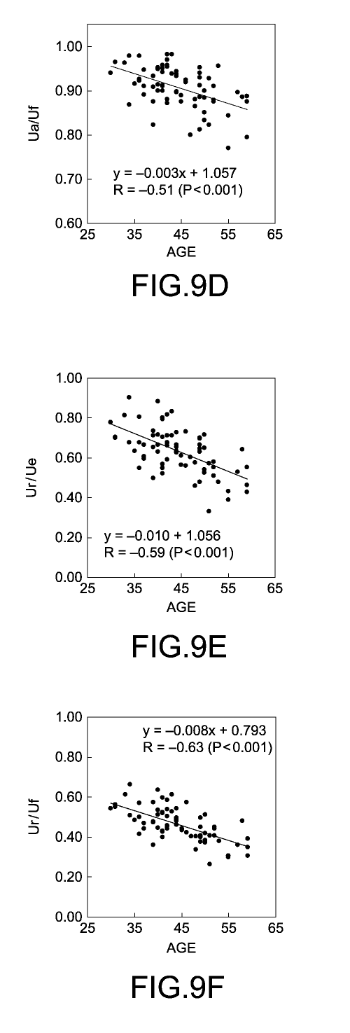

FIG. 8A to F shows the graph each representing the correlation between the facial skin elastic properties and the subcutaneous fat layer thickness. Each point represents a skin elastic property parameter and the facial subcutaneous fat layer thickness on the subject basis. The data were evaluated by calculating the Pearson's correlation coefficients. Any of the skin elastic property parameters exhibited a statistically significant negative correlation with the facial subcutaneous fat layer thickness. FIG. 9A to F shows the graph each representing the correlation between the facial skin elastic properties and the age. Each point represents a facial skin elastic property parameter and the age on the subject basis. The data were evaluated by calculating the Pearson's correlation coefficients. Any of the skin elastic property parameters exhibited a statistically significant negative correlation with the age. No correlation was observed here between the facial subcutaneous fat layer thickness and the age (data not shown). Accordingly, the increase in the facial subcutaneous fat layer thickness may be involved in the facial skin elastic properties independent with the age.

### Example 6

### Evaluation of Human Lower Cheek Sagging

The degree of sagging was evaluated in accordance with the criteria by Ezure, T. et al., (Skin Res. Technol., 15:299-305 (2009)). Briefly, the photograph of the lower cheek of a subject in a sitting position was graded as one of the six ranks based on the criteria with regard to the degree of sagging of the distended cheek region and the degree of marionette line formation.

Results

The correlation coefficient r between the facial skin elastic property parameter Ua/Ur and the lower cheek sagging degree was −0.358 with the p-value by Spearman's test being 0.002. On the other hand, the correlation coefficient r between the subcutaneous fat layer thickness and the lower cheek sagging degree was 0.442 with the p-value by Spearman's to being 1×10⁻⁴ (1×10 .sup.-4). Accordingly, both of the increase in the subcutaneous at layer thickness and the facial skin elastic property loss exhibited statistically significant correlations with the facial skin sagging.

### Example 7

### Effect of Fat Cell on Fibroblast Proliferation and Extracellular Matrix Component Production During Culturing Together

A mouse 3T3-L1 cell was employed as a fibroblast and the fat cell induced differentiation from the 3T3-L1 cell during culture was employed as a fat cell. The 3T3-L1 cell was proliferated in a Dulbecco's Modified Eagle Medium (DMEM) supplemented with 10% fetal bovine serum (FBS). The differentiation induction of the 3T3-L1 cell was conducted as described below. Thus, a 6-well multiwell plate for culturing (Cell Culture Insert/Companion plate, BD Falcon) was inoculated with 7.5×10⁴ (7.5×10 sup 4) 3T3-L1 cells per well, which was then incubated for 2 days at 37° C. in a DMEM supplemented with 10% FBS containing insulin, dexamethasone and isobutylmethyl xanthine (at final concentrations of 0.2, 0.3 and 200 micromoles, respectively). Thereafter, the cells were incubated for two days at 37° C. in DMEM supplemented with 10% FBS containing only insulin (0.2 micromoles). The 3T3-L1 cells after the differentiation induction were incubated at 37° C. in DMEM supplemented with 10% FBS. The 3T3-L1 cell induced differentiation was subjected to culturing together with the 3T3-L1 fibroblast which was not induced to differentiate on the 7th or 20th day after initiation of the differentiation induction. The 3T3-L1 fibroblast incubated in DMEM supplemented with 10% FBS without differentiation induction was inoculated in a container hanged in the wells (Cell Culture insert through which the cell cannot permeate but the culture medium components can permeate; 1.0 μm (micrometer) in pore size, 1.6×10⁶ pores/cm² (1.6×10 .sup. 6 pores/cm .sup. 2) in pore density, BD Falcon) at 3×10⁴ (3×10 .sup. 4) 3T3-L1 cells per well. The fat cell, and the fibroblast were both subjected to culturing together 12 hours after switching into DMEM supplemented with 0.5% FBS. In the control experiment, the fibroblast inoculated in the container was incubated alone in a well containing DMEM supplemented with 0.5% FBS. After the culturing together for two days, the fibroblast in the container was recovered and the cell proliferation was quantified by Alamar Blue method. For the production of the collagen, elastin and hyaluronic acid by the fibroblast, the gene expression levels of type I collagen, elastin and hyaluonic acid synthetase were quantified, respectively, by an RT-PCR method.

Results

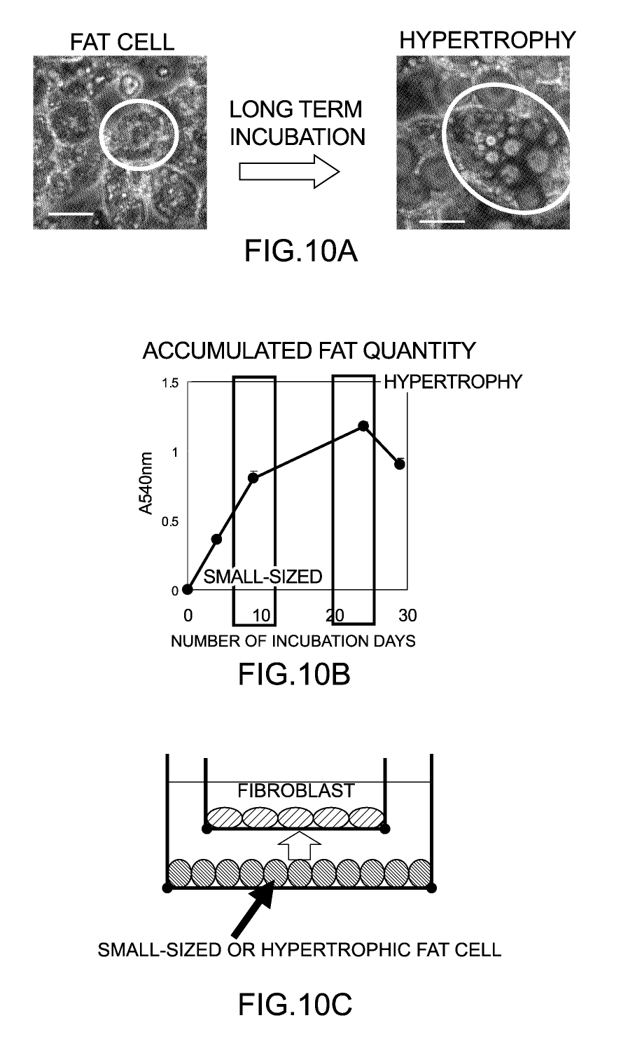

FIG. 10A is a microscopic photograph of the 3T3-L1 cell stained with Oil Red O on the 10th day (left) and the 25th day (right) after starting the differentiation induction. The region surrounded by a white line indicates a single fat cell. FIG. 10B is a graph showing the change in the accumulated fat quantity after starting the differentiation induction. As evident from FIGS. 10A and B, the fat cell was already formed on the 10th day of the differentiation induction. Thereafter, on the 25th day of the differentiation induction, the at cell became larger and the accumulated fat quantity became maximum. Hereinafter the fat cell around the 10th day of the differentiation induction is referred to as a small-sized fat cell, while the fat cell around the 25th day of the differentiation induction is referred to as a hypertrophic fat cell.

FIG. 10C is a schematic view of experiment of culturing together. The fat cell differentiated from the 3T3-L1 was incubated in each well of a multi-well plate, and a container inoculated with the non-differentiation-induced 3T3-L1 fibroblast was hanged in the each well for 2 days starting from the 7th day or the 20th day after starting the differentiation induction. The container has a membrane through which the cell, does not permeate but the culture medium component permeates. Accordingly, the fibroblast and the fat cell can undergo an interaction via a soluble factor.

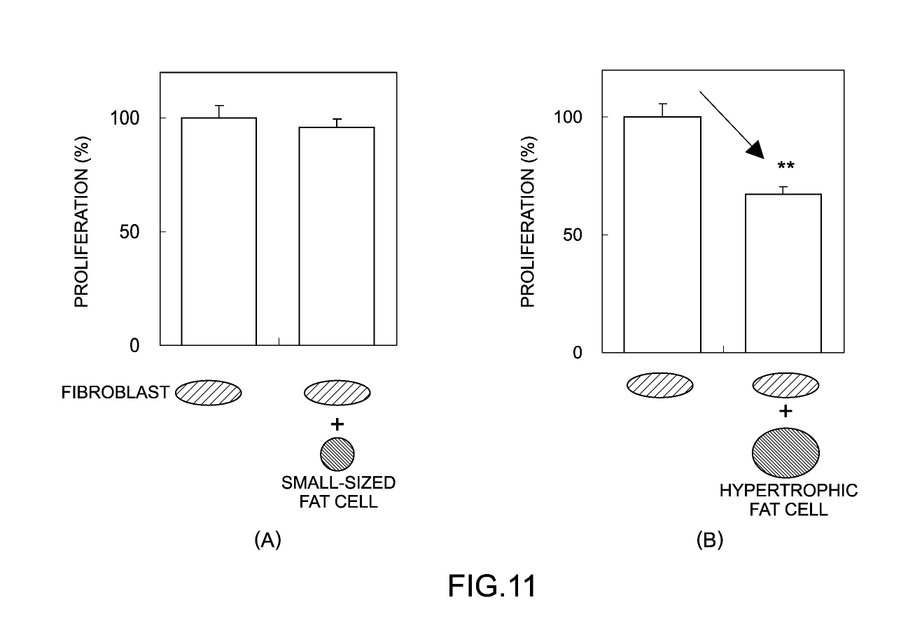

FIG. 11 is a graph showing the effect of a fat cell on the proliferation of the fibroblast during the culturing together. The % of proliferation of the fibroblast in a incubation alone and the % of proliferation of the fibroblast in culturing together with the small-sized fat cell or the hypertrophic fat cell were measured in three wells. The heights of the graph represent the averages of the % of proliferation, while the error bars in the graph indicate the standard deviations of the % of proliferation. The significant difference (**) in the average between the % of proliferation of the fibroblast in the culturing alone and the % of proliferation of the fibroblast in the culturing together with the fat cell was subjected to Student's t-test, which revealed a p-value of less than 1%. As evident from FIG. 11, the small-sized fat cell had no effect on the proliferation of the fibroblast, while the hypertrophic fat cell, exhibited the statistically significant suppression of the proliferation of the fibroblast.

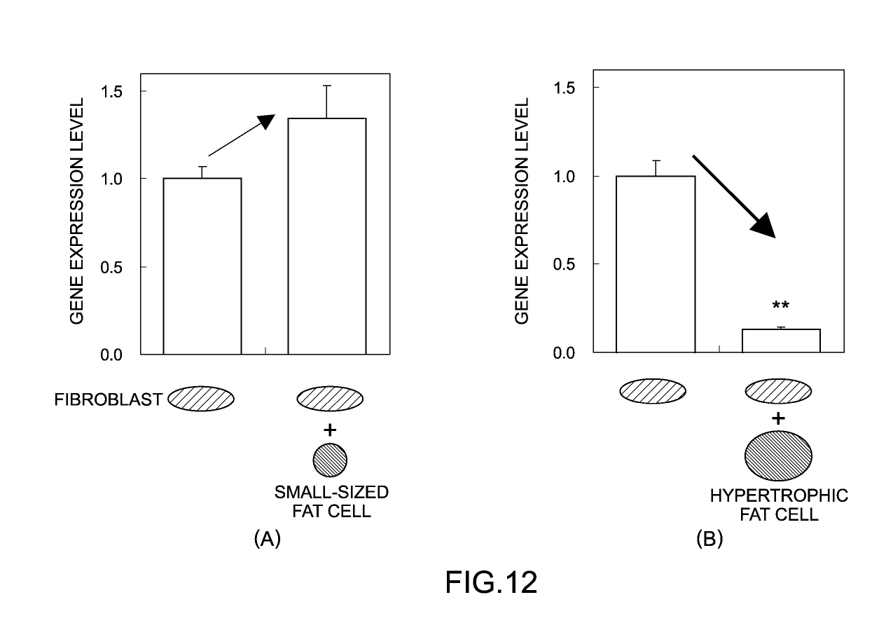

FIG. 12 is a graph showing the effect of a fat cell on the collagen production during the culturing together. The collagen gene expression level per well in culturing alone and the collagen gene expression level per well in culturing together with the fat cell were measured in three wells. The heights of the graph represent the averages of the collagen gene expression level, while the error bars in the graph indicate the standard deviations of the collagen gene expression level. The significant difference (**) in the average of the 3 wells between the collagen gene expression level per well by the fibroblast in the culturing alone and the collagen gene expression level per well in the culturing together with the fat cell was subjected to Student's t-test, which revealed p-value of less than 1%. As evident from FIG. 12, the small-sized fat cell had no effect on the collagen production, while the hypertrophic fat cell exhibited the statistically significant suppression of the collagen production.

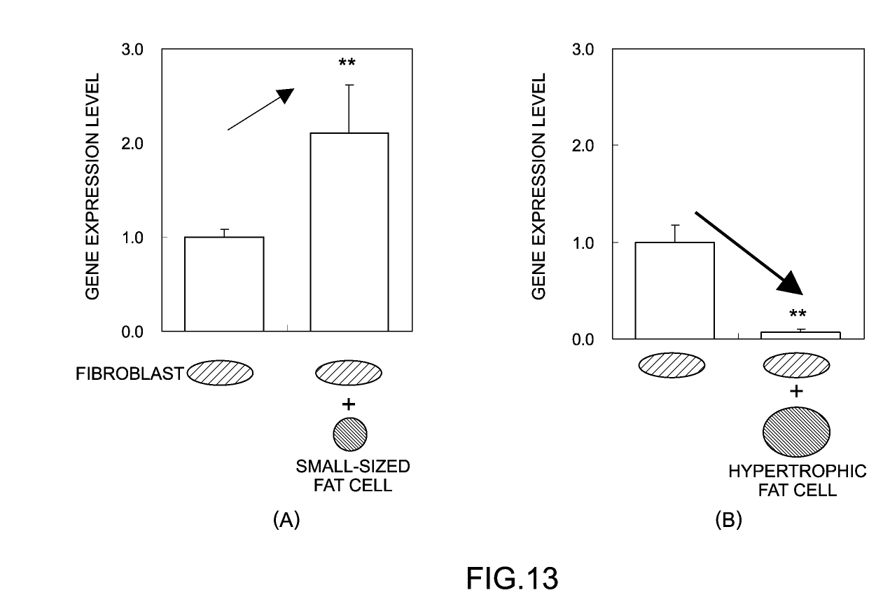

FIG. 13 is a graph showing the effect of a fat cell on the elastin production during the culturing together. The elastin gene expression level per well in a culturing alone and the elastin gene expression level per well in a culturing together with the fat cell were measured in 3 wells. The heights of the graph represent the averages of the elastin gene expression level, while the error bars in the graph indicate the standard deviations. The significant difference (**) in the average of the three wells between the elastin gene expression level per well by the fibroblast in the culturing alone and the elastin gene expression level per well in the culturing together with the at cell was subjected to Student's t-test, which revealed p—value of less than 1%. As evident, from FIG. 13, the small-sized fat cell had no effect on the elastin production by the fibroblast, while the hypertrophic fat cell exhibited the statistically significant suppression of the elastin production.

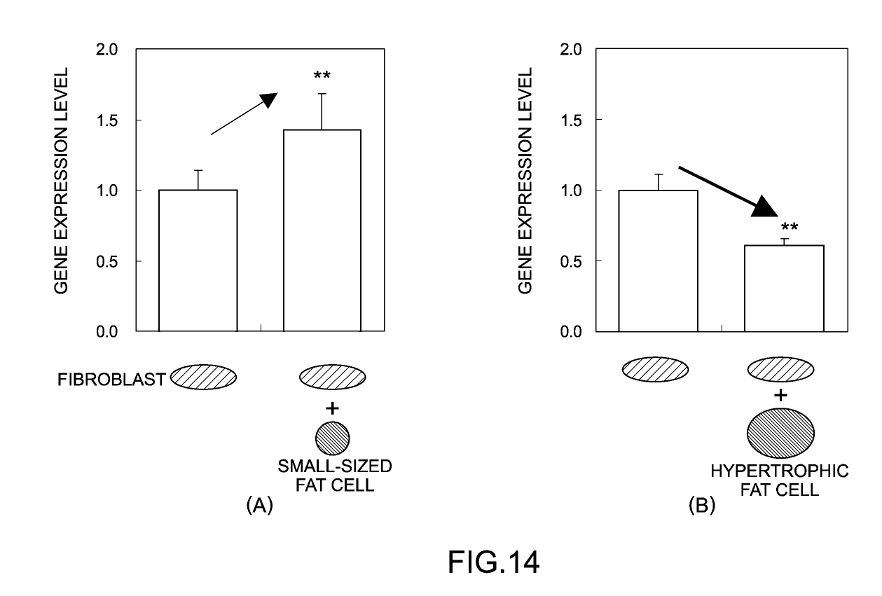

FIG. 14 is a graph showing the effect of the 3T3-L1 cell differentiation-induced into a at cell on the hyaluonic acid production by a fibroblast during the culturing together. The hyaluronic acid synthetase gene expression level per well in a culturing alone and the hyaluronic acid synthetase gene expression level per well in culturing together with the fat cell were measured in three wells. The heights of the graph represent the averages of the hyaluronic acid synthetase gene expression level, while the error bars in the graph indicate the standard deviations of hyaluronic acid synthetase gene expression level. The significant difference (**) in the average of the three wells between the hyaluronic acid synthetase gene expression level per well by the fibroblast in the culturing alone and the hyaluronic acid synthetase gene expression level per well in the culturing together with the fat cell was subjected to Student's t-test, which revealed p-value of less than 1%. As evident from FIG. 14, the small-sized fat cell had no effect on the hyaluronic acid production by the fibroblast, while the hypertrophic at cell exhibited the statistically significant suppression of the hyaluronic acid production.

Based on these results, the hypertrophic fat cell inhibited not only the proliferation of the fibroblast but also the production of the extracellular matrix components by the fibroblast. Accordingly, it is suggested that an increase in the subcutaneous fat layer thickness in a body leads to a suppression of the fibroblast proliferation in a dermis and the inhibition of the extracellular matrix production by the dermis, which results in a reduction in the dermis layer and a skin elastic property loss. In other words, by inhibiting the increase in the subcutaneous fat, a reduction in the dermis layer can be prevented. By inhibiting the increase in the subcutaneous fat, a skin elasticity loss can be prevented. By inhibiting the increase in the subcutaneous fat, an aggravation of the skin condition accompanied with a skin elasticity loss can be prevented.

1. A method for preventing a skin elastic property loss, comprising a step of suppressing an increase in a subcutaneous fat.

2. The method for preventing a skin elastic property loss according to claim 1, wherein the step of suppressing an increase in a subcutaneous fat comprises a step of applying a hyperthermic stimulation.

3. The method for preventing a skin elastic property loss according to claim 1, wherein the step of suppressing an increase in a subcutaneous fat comprises a step of administering to a subject a composition which suppresses an increase in a subcutaneous fat.

4. A cosmetic method for preventing an aggravation of a skin condition accompanied with a skin elastic property loss, comprising applying to a skin the method for preventing a skin elastic property loss according to claim 1.

5. A method for preventing a reduction in an extracellular matrix component in a dermis layer, comprising a step of suppressing an increase in a subcutaneous fat.

6. The method for preventing a reduction in an extracellular matrix component in a dermis layer according to claim 5, wherein the step of suppressing an increase in a subcutaneous fat comprises a step of applying a hyperthermic stimulation.

7. The method for preventing a reduction in an extracellular matrix component in a dermis layer according to claim 5, wherein the step of suppressing an increase in a subcutaneous fat comprises a step of administering to a subject a composition which suppresses an increase in a subcutaneous fat.

8. The method for preventing a reduction iii an extracellular matrix component in a dermis layer according to claim 5, wherein the extracellular matrix component is at least one of collagen, elastin and hyaluronic acid.

9. A cosmetic method for preventing wrinkling and sagging comprising applying to the skin a method for preventing a reduction in an extracellular matrix component in a dermis layer according to claim 5.
